(19) United States
(12) Patent Application Publication
(10) Pub. No.: US 2026/0294783 A1
(43) Pub. Date: Oct. 1, 2026

(54) COLOR COSMETICS FORMULATION

(71) Applicant: Coty Inc., New York, NY (US)

(72) Inventors: Aleksandr Plyamovaty, Brooklyn, NY (US); Tatyana Zamyatin, New York, NY (US)

(21) Appl. No.: 19/680,949

(22) Filed: May 18, 2026

Related U.S. Application Data
(63) Continuation of application No. PCT/US2024/061457, filed on Dec. 20, 2024.
(60) Provisional application No. 63/614,209, filed on Dec. 22, 2023.

(51) Int. Cl. A61K 8/891; A61K 8/06; A61K 8/37; A61Q 1/02; A61Q 19/00

(52) U.S. Cl. CPC A61K 8/891; A61K 8/064; A61K 8/37; A61Q 1/02; A61Q 19/00; A61K 2800/43; A61K 2800/48

(57) ABSTRACT

Various embodiments disclosed relate to a cosmetic formulation. The present disclosure includes a cosmetic formulation with high Longwear and good shine intensity. A formulation may include a discontinuous water phase. A formulation may include a continuous oil phase comprising: a film former comprising an MQ resin in an amount of 5% to 10% of the formulation; and an emollient system having a first component with a refractive index of at least 1.40, wherein the formulation subsequent to application to a surface demonstrates Longwear of at least 24 hours, and wherein the formulation subsequent to application to the surface demonstrates a shine intensity of at least 30 when measured by glossmeter at six hours subsequent to application to the surface.

### CROSS-REFERENCE TO RELATED APPLICATION

This application is a continuation of PCT Application Serial No. PCT/US2024/061457, filed Dec. 20, 2024, which application claims priority to U.S. Provisional Patent Application Ser. No. 63/614,209 filed on Dec. 22, 2023, which applications and publications are incorporated herein by reference in their entirety.

### BACKGROUND

Various cosmetic formulations are used by consumers to cover perceived imperfections, apply a desired color, or another purpose. For a product to be viable for consumer use, the product not only must function for its intended purpose, but it also should feel comfortable to the user. Examples of such cosmetic formulations include foundation, concealer, or other types of cosmetic formulations for application to the face or other areas.

With conventional cosmetic products, a user typically chooses between a product that is easily removable and a product that remains on the skin for long periods of time, such as an entire day. Although easily removable products can be rapidly removed by, for example, washing with soap and water, a user may find themselves repeatedly reapplying the product throughout the day, as the product may also be inadvertently removed by sweat and contact with clothing or the like. On the other hand, typical long lasting cosmetic products are less subject to inadvertent removal, but may be difficult to wash off and may require the use of organic solvents or other harsh cosmetic removers.

### SUMMARY OF THE DISCLOSURE

In some aspects, the techniques described herein relate to a cosmetic formulation including a water in oil emulsion for application to an integument, wherein the cosmetic formulation includes: a discontinuous water phase; and a continuous oil phase including: a film former including an MQ resin in an amount of 5% to 10% of the formulation; and an emollient system having a first component with a refractive index of at least 1.40; wherein the formulation subsequent to application to a surface demonstrates Longwear of at least 24 hours, and wherein the formulation subsequent to application to the surface demonstrates a shine intensity of at least 30 when measured by glossmeter at six hours subsequent to application to the surface.

In some aspects, the techniques described herein relate to a cosmetic formulation including a water in oil emulsion for application to an integument, wherein the cosmetic formulation includes: An oil phase including: a film former including an MQ resin, the film former in an amount of 5.0% to 10.0% of the formulation; and an emollient system having the emollient system being in an amount of 15.0% to 45.0% of the formulation, the emollient system including a first component with a refractive index of at least 1.40, a second component, and optionally a third component; a thickener in an amount of 1.5% to 5.0% of the formulation; pigments in an amount of 10.0% to 20.0% of the formulation; a powder in an amount of 1.5% to 5.0% of the formulation; and an emulsifier in an amount of 2.0% to 8.0% of the formulation; and a water phase in an amount of 10.0% to 25.0% of the formulation.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a graph of shine intensity testing results in an example.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

The present disclosure describes, among other things, a cosmetic formulation using a specific film former, a Trimethylsiloxysilicate resin, in combination with an emollient system including Diphenylsiloxy Phenyl Trimethicone to deliver Longwear, dewiness, and coverage across a wide variety of skin types with beneficial viscosity.

The cosmetic formulation is a water in oil emulsion for application to an integument. The emulsion includes both a discontinuous water phase and a continuous oil phase. The oil phase includes a film former including an MQ resin, the emollient system having a high reflective index, and several pigments, in addition to other components such as a thickener, silica, and story ingredients. The water phase includes various water-soluble components, such as glycols.

Generally, cosmetic formulations have either provided a “dewy” finish or provided Longwear to the consumer. A dewy finish can, for example, provide a radiant, luminous look that gives the appearance of a healthy, glowing complexion. This is often lost in Longwear cosmetics formulations. A Longwear formulation can be, for example, a cosmetics formulation that will last for hours, or days, such as up to 24 or 36 hours.

Moreover, providing a long wear formulation that is comfortable for all skin types can be challenging. While oily skin types may be better adapted to Longwear formulations from a comfort perspective, dry skin types may result in dried-out Longwear cosmetic layers that cake or flake. Conversely, oily skin types may break down Longwear formulations quicker by producing more sebum compared to dry skin types. Combination skin types can be challenged with a combination of these issues. Formulating a Longwear cosmetic formulation that works well across the spectrum to provide a beneficial look, feel, and wear, can be challenging.

The cosmetic formulations discussed herein allow for a longwear formulation that also provide a dewy finish, delivering long lasting coverage while maintaining comfort on the user's skin. The cosmetic formulations discussed herein can be used successfully on a wide variety of skin types, including dry, oily, and combination skin. The cosmetic formulations discussed herein can be adaptive to the skin type and to varying environmental conditions. Overall, the cosmetic formulations discussed herein can produce beneficial longwear, viscosity, dewiness, and coverage, among other beneficial properties.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In the event of inconsistent usages between this document and any documents so incorporated by reference, the usage in this document controls.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substantially free of” as used herein can mean having none or having a trivial amount of, such that the amount of material present does not affect the material properties of the formulation including the material, such that about 0 wt % to about 5 wt % of the formulation is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

### Cosmetic Formulation

According to various aspects of the present disclosure, a cosmetic formulation can deliver desired longwear coverage. The cosmetic formulation can have a desired combination of a film former with a specific emollient system to deliver beneficial properties across a variety of skin types.

The cosmetic formulation can be a water in oil emulsion for application to an integument (e.g., skin of a consumer), such as for use as a foundation. The cosmetic formulation can include an MQ resin film former and an emollient system having at least one component with a minimum refractive index.

The cosmetic formulations discussed herein can be an emulsion. The emulsion can have a viscosity of 10,000 cP to 20,000 cP as measured via Brookfield viscosity with a TB spindle at a speed of 10 rpm for 1 minute. The emulsion can have a minimum viscosity of about 10,000 cP, or of about 11,000, 12,000, 13,000, 14,000, 15,000, 16,000, 17,000, 18,000, 19,000 or about 20,000 cP.

The cosmetic formulation can have, once applied to an integument (e.g., a user's skin or other surface) a Longwear of at least 24, or a Longwear of about 24 hours, 26, 27, 28, 29, 30, 31, 32, 33, 34, or about 36 hours.

The cosmetic formulation can have, once applied to an integument (e.g., a user's skin or other surface) a shine intensity of at least 40 when measured by 60-degree reflection at six hours. In an example, the cosmetic formulation can have a shine intensity of about 30 to 50, or about 30, 31, 32, 33, 34, 35, 36, 37, 38, 39, 40, 41, 42, 43, 44, 45, 46, 47, 48, 49, or about 50.

The cosmetic formulation can have, once applied to an integument (e.g., a user's skin or other surface) a Crockmeter dry measurement of about 6.5 to 8.5, or of about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or about 8.5.

The cosmetic formulation can have, once applied to an integument (e.g., a user's skin or other surface) a Crockmeter squalene measurement of about 14.0 to about 16.0, or about 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, or about 16.0.

The cosmetic formulation can have, once applied to an integument (e.g., a user's skin or other surface) a Crockmeter water measurement of about 3.5 to about 4.5, or of about 3.5, 3.6, 3.7, 3.8, 3.9, 4.0, 4.1, 4.2, 4.3, 4.4, or about 4.5

The cosmetic formulation can have, once applied to an integument (e.g., a user's skin or other surface, a Crockmeter saline measurement of about 4.0 to about 5.0, or of about 4.0, 4.1, 4.2, 4.3, 4.4, 4.5, 4.6, 4.7, 4.8, 4.9, or about 5.0.

The cosmetic formulation can have a stability of greater than 1 week, or of greater than about 1, 2, 3, 4, 5, 6, 7, 8, 9, 10, 11, 12, 13, 14 or about 15 weeks.

The cosmetic formulation can have an example formulation as summarize in Table 1 below:

TABLE 1
Example cosmetic formulation.

| Phase | Component | Examples | Amount |
|---|---|---|---|
| Oil Phase | Film former | TMS | 5.0-15.0% |
| | Emollients | Dimethicone | 15.0-45.0% |
| | | Diphenylsiloxy Phenyl | |
| | | Trimethicone | |
| | | Diisopropyl sebacate | |
| | | Silicon fluid | |
| | Emulsifier | Cetyl Diglyceryl | 2.0-8.0% |
| | | Tris(Trimethyl- | |
| | | siloxy)silylethyl | |
| | | Dimethicone | |
| | Powder | Silica | 1.0-5.0% |

| | |
|---|---|
| Thickeners | 1.5-5.0% |
| Pigments | 10%-20% |

| | |
|---|---|
| Water Phase | 10.0-25.0% |

Additional components as desired

Oil Phase. The oil phase of the cosmetic formulation can include a variety of components, including but not limited to film formers, emollients, emulsifiers, vitamins, pigments, thickeners, story ingredients, active components, and other components.

Film formers. The oil phase of the cosmetic formulation can include one or more film formers. The film formers can aid in the formation of a flexible and non-transferring film, and help prevent transfer while increasing wear of the formulation. The film formers can include, for example, polymers.

The film formers can include, for example, a silicone MQ resin. An MQ resin is a silicone resin with a unique structure. MQ resins have of mono-(M) and tetra-(Q) functional siloxane units that occupy an important place among organosiloxane polymers. In an example, the MQ resin can be trimethylsiloxysilicate (TMS).

The film former can be provided in an amount of about 5% to about 10% of the formulation, or of about 5.0, 5.5, 6.0, 6.5, 7.0, 7.5, 8.0, 8.5, 9.0, 9.5, or about 10.0% of the formulation.

Emollient system. The formulation can include an emollient system, such as to aid in slowing evaporation of water from the formulation and to provide enhanced application.

The total amount of emollients in the formulation can be about 15% to about 35% 45% or of about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45% of the formulation.

The emollient system can include at least one emollient that has a high refractive index. For example, the emollient with a high refractive index can have a refractive index of about 1.35 or higher, or of about 1.35, 1.40, 1.45, 1.50, 1.55, 1.60, or higher. The emollient with a high refractive index can be about 10% to about 35% of the emollient system, or of about 10, 11, 12, 13, 14, 15, 16, 17, 18, 19, 20, 21, 22, 23, 24, 25, 26, 27, 28, 29, 30, 31, 32, 33, 34, or about 35% of the emollient system. The emollient with a high refractive index can be, for example, diphenylsiloxy phenyl trimethicone.

The emollient system can optionally additionally include other emollients, such as petrolatum, paraffin, mineral oil, glycerin, beeswax, olive oil, coconut oil, lanolin, cocoa butter, synthetics such as butyl stearate and diglycol laurate, dimethicone, diisopropyl sebacate or combinations thereof.

In an example, the emollient system of the cosmetic formulation can include three components: dimethicone, diphenylsiloxy phenyl trimethicone, and diisopropyl sebacate. In an example, the dimethicone can be about 15% to about 20% of the formulation. In an example diphenylsiloxy phenyl trimethicone can be about 5% to about 25% of the formulation. In an example diisopropyl sebacate can be about 3% to about 6% of the formulation.

Emulsifiers. The formulation can include an emulsifier to aid in mixing of the water phase and the oil phase. An emulsifier is particularly suitable when the cosmetic formulation is in the form of an emulsion or if immiscible materials are being combined. The emulsifier can have micellar properties, lamellar properties, or both. Emulsifiers may be nonionic, anionic, zwitterionic, or canonic.

In an example, the emulsifier can be about 2.0 to about 8.0 wt % of the formulation, or about 2.0, 2.5, 3.0, 3.5, 4.0, 4.5, 5.0, 5.5, 6.0, 6.5, 7.0, 7.5, or 8.0 wt % of the formulation.

In an example, the emulsifier can include Glyceryl Stearate Citrate, Polyglyceryl-3 Stearate, and Hydrogenated Lecithin, PEG-10 Dimethicone, Lauryl Polylglyceryl-3 Polydimethylsiloxyethyl Dimethicone, Cetyl PEG/PPG-10/1 Dimethicone, or combinations thereof.

Pigments. The formulation can include one or more pigments. The pigments, such as yellow, red, black, brown, or other pigments, can be used for coloration, such as to provide a desired skin shade for the formulation. In some cases pearls or glitters can be used.

The instantly disclosed formulation can include a colorant or pigment component, which can be present in the formulation in a range of from about 5 wt % to about 20 wt % of the formulation, about 8 wt % to about 12 wt % of the formulation, less than, equal to, or greater than about 5 wt %, 10, 15, or 20 wt % of the formulation.

Colorants suitable for use in the present formulations include, but are not limited to, dyes, pigments, lakes, and mixture thereof. (e.g., organic or inorganic pigments and colorants approved for use in face area cosmetics by CTFA and/or the FDA.) Exemplary inorganic pigments include particles of iron oxides (e.g., yellow, brown, red, black), titanium dioxides, iron sulfides, ultramarines, chromium oxides (e.g., green) or other conventional pigments used in cosmetic formulations. Examples of organic pigments include FD&C Red No. 40, D&C Green No. 5, FD&C Blue No. 1, and FD&C Yellow No. 5, FD&C Red No. 6, FD&C Red No. 27, FD&C Red No. 28, FD&C Yellow No. 6, and others. Examples of lake dyes include various acid dyes which are laked with aluminum, calcium or barium. Additional colorants for use herein include annatto, caramel, carmine, β-carotene, bismuth oxychloride, ferric ammonium ferrocyanide, ferric ferrocyanide, chromium hydroxides (e.g., green), guanine, mica, aluminum powder, bronze powder, copper powder, manganese violet, zinc oxide. Suitable colorants along with their chemical structure are described in, e.g., 21 C.F.R. Part 74 and in the CTFA Cosmetic Ingredient Handbook, (1988), published by the Cosmetics, Toiletry and Fragrances Association, Inc. Other colorants may also be used as they are developed and determined safe.

Thickener. The formulation can include one or more thickeners or gellants in the water phase, oil phase, or both. The thickeners or gellants can aid in the viscosity and tack of the formulation, and aid in coverage.

The thickeners or gellants can include, for example, clay, gum, or cetyl alcohol. In an example, the thickener can include tapioca starch or pure tapioca. In an example, the thickener can include Stearalkonium Bentonite. In an example, the thickener can be about 1.5% to about 5.0% of the formulation, or about 1.5, 2.0, 2.5, 3.0, 3.5, 4.0, 4.5, or about 5.0% of the formulation.

A variety of oil soluble solvents can be used in the oil phase, such as propylene carbonate or others.

Water Phase. The water phased can include a variety of additional components that are water soluble, such as active ingredients and story ingredients discussed below. A variety of solvents can be in the water phase, such as, for example, water, glycerin, and aqueous solutions such as sodium chloride solution. In an example, the water phase can be about 10% to about 25% of the formulation, or about 10, 11, 12, 13, 14, 15, 16, 17, 18, 19, 20, 21, 22, 23, 24, or about 25% of the formulation.

Active Components. A variety of active components can be in the oil phase, the water phase, or both. For example, the formulation can include moisturizing agent, emollient, filler, coloring agent, essence or spice, skin conditioner, vitamin, light protective agents, antiperspirant, oil control, antioxidant, crease-resistant material and other suitable materials.

In an example, the active ingredients can include vitamins, such as vitamin E or niacinamide. In an example, the active ingredients can include moisturizing agents, such as 2,3-butanediol, Acid, 1-2 Hexanediol, Caprylyl Glycol, or others. In an example, the active ingredients can include UV protectors, such as zinc oxide, Triethoxycaprylylsilane, and others. In an example, the active ingredients can include fungicidal agents, such as chlorphenesin. In an example, additional active components can include preservatives.

Other Components. The formulation can optionally include other components in the oil phase, the water phase, or both.

Example additional components can include powders, rheology modifying components, or story ingredients. A variety of powders, such as water dispersible powders, oil dispersible powders, or a combination, can be used. For example, non-absorbent powders such as silica can be included, or absorbent powders can be included. Silica can be included, for example, in an amount of about 1.5% to about 5.0%. Silica can act as a sponge in the formulation. In some cases, powders such as titanium dioxide can be included.

A variety of story ingredients can be included in the formulation, including but not limited to conditioning agents, butter, functional ingredients, fragrance, and others. For example, fragrance components such as polysorbate or ethylene brassylate can be used.

Other additives may also be included. The formulation can further include an oil-soluble or dispersible additive which may include fatty alcohols such as Guerbet alcohols based on fatty alcohols having from 6 to 30, preferably from 10 to 20 carbon atoms including lauryl alcohol, cetyl alcohol, stearyl alcohol, cetearyl alcohol, oleyl alcohol, benzoates of C₁₂-C₁₅ alcohols, acetylated lanolin alcohol, etc. Especially suitable is stearyl alcohol. The oil-soluble or dispersible additive may include fatty acids such as Linear fatty acids of C₆-C₂₄, branched C₆-C₁₃carboxylic acids, hydroxycarboxylic acids, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids).

Further components that can be used are dicarboxylic acids of C₂-C₁₂, such as adipic acid, succinic acid, and maleic acid. Aromatic carboxylic acids, saturated and/or unsaturated, especially benzoic acid, can be used. Additional components that can be used as the oil soluble or dispersible additive include carboxylic acid salts; alkaline soaps of sodium, potassium and ammonium; metallic soaps of calcium or magnesium; organic basis soaps such as lauric, palmitic, stearic and oleic acid, etc., alkyl phosphates or phosphoric acid esters: acid phosphate, diethanolamine phosphate, potassium cetyl phosphate.

Other useful oil-soluble or dispersible additives comprise mild surfactants, super-fatting agents, consistency regulators, additional thickeners, polymers, stabilizers, biologically active ingredients, deodorizing active ingredients, oil control agents film formers, swelling agents, UV light-protective factors, antioxidants, preservatives, insect repellents, solubilizers, colorants, bacteria-inhibiting agents, conditioning agents, vitamins, and the like.

There are any number of further components that can be included in the formulation such as a surfactant component, a skin conditioner component, a humectant component, or another additive component. Other additives can include chamomile and cucumber extract, jojoba oil, camellia oil, aloe butter, maracuja oil, marula oil, castor oil, ceramide 2, avocado butter, or a mixture thereof.

Skin Conditioning Agent. The cosmetic formulation can optionally include additional skin conditioning ingredients or agents, such as Propanediol, Glycerin, Nymphaea Caerulea Flower Extract, and Nelumbo Nucifera Flower Extract.

For example, such a skin conditioning agent can include extracts from lotus flowers, the Egyptian Lotus (Nymphaea caerulea) and the Sacred Lotus (Nelumbo nucifera). The use of these two extracts together can help synergistically transmit luminosity, reduce inflammation, reduce perspiration, and/or reduce sebum production.

In an example, the cosmetic formulation can include about 0.2% to about 2.0% of the lotus flower extract, or about 0.2, 0.3, 0.4, 0.5, 0.6, 0.7, 0.8, 0.9. 1.0, 1.1, 1.2, 1.3, 1.4, 1.5, 1.6, 1.7, 1.8. 1.9, or about 2.0% of the formulation.

In one aspect, the lotus plant derived constituents may be formulated and/or utilized in products and/or regimens that reduces perspiration in a subject. In some examples, perspiration reduction is accomplished primarily without clogging pores.

In one aspect, the lotus plant derived constituents may be formulated and/or utilized in the form of a primer for creating a substantially flawless canvas for longer lasting makeup following application. In another embodiment, the lotus plant derived constituents may be formulated and/or utilized in the form of a foundation composition. Suitable foundation formulations are those that are capable of evening out skin tone, concealing imperfections and/or providing a flawless matte finish.

In another aspect, the lotus plant derived constituents may be formulated and/or utilized in the form of a cosmetic concealer that substantially conceals small imperfections, dark spots and circles.

In another aspect, the lotus plant derived constituents may be formulated and/or utilized in the form of combination cosmetic foundation/concealer product. In another aspect, the lotus plant derived constituents may be formulated and/or utilized in the form of a setting spray that may be sprayed over make-up to substantially lock in a look (following make-up application) and protect the skin. The lotus plant derived constituents may be utilized to help reduce breakouts caused by make-up and sweat.

Additional skin conditioning agents can include bisabolol, propylene carbonate, 2,3-Butanediol, pentylene glycol, fructose, urea, citric acid, sodium hydroxide, maltose, sodium PCA, sodium lactate, sodium chloride, trehalose, allantoin, sodium hyaluronate, glucose, physiogenyl powder, zinc PCA, manganese PCA, magnesium PCA, 3-O-ethyl ascorbic acid, panthenol, cyclopentasiloxane, dimethiconol, and various other components in various combinations as desired.

In one embodiment, a skin conditioning ingredient trademarked NELUPURE™ is utilized. This material is a vegetal complex containing flower extracts of Nelumbo nucifera and Nymphaea caerulea. NELUPURE as herein described is comprised of the following components:

Method of Making Cosmetic Formulation

| Ingredient | Percentage | CAS | EINECS |
|---|---|---|---|
| Propanediol | 70-90% | 504-63-2 | 207-997-3 |
| Glycerin | 10-30% | 56-81-5 | 200-289-5 |
| Nymphaca Caerulea | 0.5-1% | — | — |
| Flower Extract | | | |
| Nelumbo Nucifera | 0.5-1% | 85085-51-4 | 285-379-2 |
| Flower Extract | | | |

The instantly described cosmetic formulations can be made according to a variety of suitable methods. As an example, water and the ingredients in the water phase can be added to a beaker and exposed to low shear conditions (about 100 rpm) at room temperatures (about 25° C.). Other components such as pigments, emulsifiers, and the like can be added to create the oil phase. The water phase and the oil phase can be combined with the emulsifier to create the formulation.

### Method of Using

The cosmetic formulation can be contained in an applicator assembly. A variety of conventional applicators are known in the art (e.g., sponges, brushes, wipes, blenders, or other applicators). Optionally, a user can apply the cosmetic formulation by hand. Whether applied by an applicator or by hand, the cosmetic formulation can be applied directly to the skin. The cosmetic formulation can be applied in a single layer, or in multiple layers to create the desired effect. The cosmetic formulation can be blended and shaped by the user on the skin to create the desired coverage.

Optionally, the cosmetic formulation can be used with a variety of other cosmetic products, such as but not limited to skin care compositions, other foundations or concealers, other types of color cosmetic formulations such as blush, bronzer, or highlighter, among others. Each of these can be combined, blended, or adjusted during applicator before, after, or simultaneously with the cosmetic formulation discussed herein, to provide the desired makeup look.

### EXAMPLES

### Example 1. Cosmetic Formulation

For each of the Examples below, a sample cosmetic formulation was produced with the following formulations, except where otherwise noted.

TABLE 2A
Formulation A.

| Component | Type/Source | Amount |
|---|---|---|
| Film Formers | Belsil TMS-803 (Wacker Chemie AG) | 8% |
| Emollients | SeraSence 1.5 (KCC Beauty) | 41% |
| | KF-56A (Shin-Etsu Silicones) | |
| | Shercemol DIS ester (Lubrizol) | |
| Emulsifier | Dowsil ES 5600 (Dow) | 5% |

TABLE 2B
Formulation B.

| Component | Type/Source | Amount |
|---|---|---|
| Film Formers | Belsil TMS-803 (Wacker Chemie AG) | 8% |
| Emollients | XIAMETER PMX-200 SILICON | 42% |
| | FLUID 1.5 cP (Dow Chemical) | |
| | KF-56A (Shin-Etsu Silicones) | |
| | Shercemol DIS ester (Lubrizol) | |
| Emulsifier | KF-6017 (Shin-Etsu Silicones of | 5% |
| | America) | |
| Powder | MSS-500W (Kobo Products, Inc.) | 2% |
| Skin Conditioning | NELUPURE | 0.5% |
| Agent | | |

TABLE 2C
Formulation C.

| Component | Type/Source | Amount |
|---|---|---|
| Film Formers | Belsil TMS-803 (Wacker Chemie AG) | 8% |
| Emollients | XIAMETER PMX-200 SILICON | 42% |
| | FLUID 1.5 cP (Dow Chemical) | |
| | KF-56A (Shin-Etsu Silicones) | |
| | Shercemol DIS ester (Lubrizol) | |
| Emulsifier | KF-6017 (Shin-Etsu Silicones of | 5% |
| | America) | |
| Powder | MSS-500W (Kobo Products, Inc.) | 2% |

Other components not listed in Tables 2A to 2C were held consistent across the three formulations. These components included vitamins, skin conditioning agents, fragrances, pigments, solvents, thickeners, and other story ingredients.

### Example 2. Crockmeter, Viscosity, and Stability

Three samples of the cosmetic formulation with various film formers were prepared and tested for wear, viscosity, and stability, through a variety of testing protocols. These samples are summarized below:

TABLE 3
Sample formulations with film formers.

| ID | Film Former | Type/Source |
|---|---|---|
| A | 8% TMS | Belsil TMS-803 (Wacker Chemie AG) |
| B | 8% Phenylpropyl- | SilShine 151 (Momentive |
| | dimethylsiloxysilicate | Performance Materials) |
| C | 8% TMS | Belsil TMS-803 (Wacker Chemie AG) |
| | 2% Ammonium Acrylate | Micromer C 60/41 (Worlee Chemie |
| | Copolymer | GmbH) |

The Sample A included trimethylsiloxysilicate (“TMS”), an MQ resin. Sample B, for comparison, included phenylpropyldimethylsiloxysilicate, a siloxane polymer. Sample C include the TMS, but additionally included water-soluble acrylate copolymers. These three samples were formulated that same with the exception of the film formers.

Each of the samples were tested with the Crockmeter for wear, for viscosity, and observed for stability. The results of these tests are summarized below:

TABLE 4
Sample testing results.

| | Crockmeter | | | | Viscosity | |
|---|---|---|---|---|---|---|
| ID | DRY | SQUALANE | WATER | SALINE | (cP) | Stability |
| A | 7.2 | 14.7 | 3.8 | 4.3 | 14,240 | Stable |
| | | | | | | 15 weeks |
| B | 5.6 | 14.7 | 3.8 | 4.0 | 13,720 | Unstable |
| | | | | | | 3 weeks |
| C | 4.1 | 13.4 | 2.8 | 3.4 | 5,920 | Unstable |
| | | | | | | 1 weeks |

These Samples were tested for wear (or “colorfastness”) in a rub test with a Crockmeter under a modified AATCC method 116-2013-Colorfastness to Crocking. Several different rubbing insult types are tested: rub testing as a dry rub, with water, saline, and artificial sebum (squalene). Each of these liquids represents a different liquid that a user might come into contact with throughout the day while wear the formulation. Each liquid (water, saline, squalene) could potentially move the formulation from the user's skin. Moreover, some of these liquids, such as water, could potentially be used at the end of the day to remove the formulation from the user's face. Preferably, the formulation does not transfer or mover from the surface.

For the rub testing, each of the samples was prepared on linen. One each of the dry, water, saline, and squalene slides were produced with each of the Samples. For the wet samples, a small 20 μL drop of the liquid (water, saline, or squalene) was pipetted onto the sample. The samples were then put into the Crockmeter according to the AATCC method. A manual crank of 10 was used. The rub testing of the samples on the Crockmeter was iterated.

The results of the Crockmeter testing of samples are depicted above in Table 4. A very low Crockmeter value can indicate the sample was difficult to remove, while a very high Crockmeter value can indicate the sample was prone to transferring. Overall, the Sample A with the TMS performed the best across the various Crockmeter tests.

These Samples were tested for viscosity on a Brookfield viscometer with a TB spindle at 10 rpm for one minute. The resulting viscosity measurements are shown in Table 4 above. Overall, the Sample A with the TMS performed well.

These Samples were observed for stability at one week, three weeks, and fifteen weeks after production. Stability was assessed visually after the formulation was left to sit. Stability was evaluated at room temperature, at 5° C., 37° C., 45° C., and at freeze and thaw. Stability of the Sample C with water dispersible polymers was bad after only one week. Stability of the Sample B with a different film former was bad after 3 weeks. The Sample A with the TMS performed well, and was still stable at 15 weeks.

Overall, the Sample A using the TMS film former performed the best in the combined parameters of wear (Crockmeter), viscosity, and stability.

### Example 3. Dewiness

Two samples of the cosmetic formulation with various emollients were prepared and tested for dewiness or “shine intensity” through use of a glossmeter. These samples are summarized below:

TABLE 5
Sample formulations with emollients.

| ID | Emollient System | Type (Source) |
|---|---|---|
| D | Dimethicone | SeraSence 1.5 (KCC Beauty) |
| | Diisopropyl sebacate | SCHERCEMOL DIS |
| | Diphenylsiloxy Phenyl | KF-56A (Shin-Etsu) |
| | Trimethicone | |
| E | Dimethicone | SeraSence 1.5 (KCC Beauty) |
| | Diisopropyl sebacate | SCHERCEMOL DIS (Lubrizol) |

The Samples D and E were tested for shine intensity with the use of a BYK-GARDNER glossmeter. The samples were applied in 6 mil with an Applicator (Bird) to a coated card (LENETA, form 5C). The drawdown of the samples were made, and the glossmeter was placed on the drawdown.

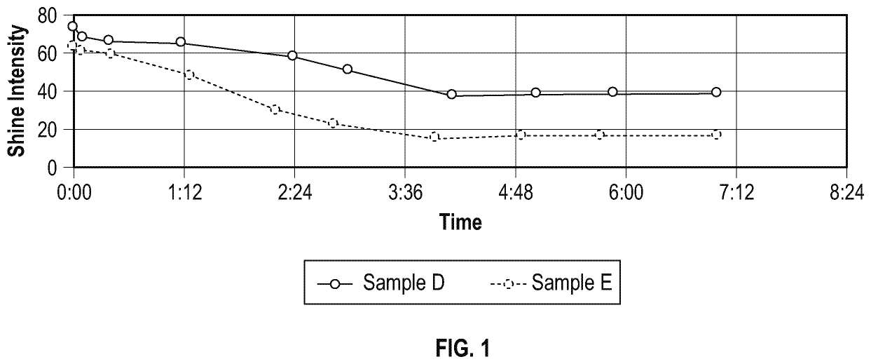

The gloss of the sample was measured with the glossmeter through standard BYK-GARDNER glossmeter procedure. The gloss intensity was measured at 20, 60, and 85 degrees. These measurements were taken at predetermined time intervals after preparation of the samples, from initial application through about seven hours after application. At each measurement, the time elapsed since initial application was recorded. FIG. 1 depicts the results of the glossmeter testing with a 60 degree reflection.

Overall, the Sample D which included diphenylsiloxy phenyl trimethicone in the emollient system performed much better than Sample E without. Sample D had a higher shine intensity that lasted longer.

### Example 4. Coverage and Longwear

The inventive sample (as summarized in Table 2) was also tested in Consumer Sensory testing for Longwear and coverage against a commercially available competing foundation product.

Here, panelists came in with a clean face, and a baseline photo of each panelist's face (e.g., clean with no cosmetics) was taken. The sample cosmetic formulation was applied as foundation to the panelists' faces with their fingers, avoiding the eye area. The cosmetic formulation was allowed five minutes to dry. Then, a subsequent photo was taken to observe coverage of the cosmetic formulation.

The panelists were then sent home to wear the applied cosmetic formulation for 24 hours. The panelists were instructed to avoid washing their face, and were allowed one shower, avoiding the face, without hair washing. They were instructed to avoid water streaming down the face, and allowed “bird bath” style washing, with air drying of the face. No face washing, skincare, SPF, swimming, sauna, other makeup or other products were allowed for the panelists during this time period.

The panelists were asked to analyze the cosmetic formulation in the mirror in the evening before bed and in the morning. The panelists were asked to see if they observed any product left on their skin.

The panelists were asked a series of questions, including the following:

TABLE 6
Sensory testing questions.
Questions

Q: Is it still on? If no, how many hours did it last?
Q: Did you shower?
Q: What did they did over the 24 H (gym, exercising, etc.)?
Q: Self Identify skin type (oily, dry, combination, etc.)

Photos were taken. The cosmetic formulation was removed with a folded wipe. Images of the panelists' faces were taken after removal. Images of the remover wipe were also taken. Overall, the inventive sample produced good Longwear, with a majority of product still present after 24 hours of wear.

### Various Notes & Examples

In some aspects, the techniques described herein relate to a cosmetic formulation including a water in oil emulsion for application to an integument, wherein the cosmetic formulation includes: a discontinuous water phase; and a continuous oil phase including: a film former including an MQ resin in an amount of 5% to 10% of the formulation; and an emollient system having a first component with a refractive index of at least 1.40; wherein the formulation subsequent to application to a surface demonstrates Longwear of at least 24 hours, and wherein the formulation subsequent to application to the surface demonstrates a shine intensity of at least 30 when measured by glossmeter at six hours subsequent to application to the surface.

In some aspects, the techniques described herein relate to a cosmetic formulation, wherein the formulation has a viscosity of 10,000 cP to 20,000 cP as measured via Brookfield viscosity with a TB spindle at 10 rpm for 1 minute.

In some aspects, the techniques described herein relate to a cosmetic formulation, wherein the formulation includes a stability of at least 8 weeks.

In some aspects, the techniques described herein relate to a cosmetic formulation, wherein the formulation subsequent to application to a surface demonstrates Longwear of at least 36 hours.

In some aspects, the techniques described herein relate to a cosmetic formulation, wherein the formulation subsequent to application to the surface demonstrates a shine intensity of 30 to 50 when measured by glossmeter at six hours subsequent to application to the surface.

In some aspects, the techniques described herein relate to a cosmetic formulation, wherein film former includes 5% to 10% of the emulsion.

In some aspects, the techniques described herein relate to a cosmetic formulation, wherein film former includes trimethylsiloxysilicate.

In some aspects, the techniques described herein relate to a cosmetic formulation, wherein the emollient system includes about 15% to about 45% of the emulsion.

In some aspects, the techniques described herein relate to a cosmetic formulation, wherein the first component of the emollient system includes 10% to 20% of the emollient system.

In some aspects, the techniques described herein relate to a cosmetic formulation, wherein the first component of the emollient system includes diphenylsiloxy phenyl trimethicone.

In some aspects, the techniques described herein relate to a cosmetic formulation, wherein the emollient system further includes diisopropyl sebacate, dimethicone, and combinations thereof.

In some aspects, the techniques described herein relate to a cosmetic formulation, wherein the oil phase further includes an emulsifier being 2.0% to 8.0% of the formulation.

In some aspects, the techniques described herein relate to a cosmetic formulation, wherein the oil phase further includes a thickener.

In some aspects, the techniques described herein relate to a cosmetic formulation, wherein the oil phase further includes one or more pigments including yellow pigments, red pigments, black pigments, white pigments, or combinations thereof.

In some aspects, the techniques described herein relate to a cosmetic formulation, wherein the discontinuous water phase includes a moisturizing ingredient.

In some aspects, the techniques described herein relate to a cosmetic formulation, further including an absorbent powder in an amount of 1.5% to 5.0%.

In some aspects, the techniques described herein relate to a cosmetic formulation, further including Nymphaea Caerulea Flower Extract and Nelumbo Nucifera Flower Extract.

In some aspects, the techniques described herein relate to a cosmetic formulation including a water in oil emulsion for application to an integument, wherein the cosmetic formulation includes: An oil phase including: a film former including an MQ resin, the film former in an amount of 5.0% to 10.0% of the formulation; and an emollient system having the emollient system being in an amount of 15.0% to 45.0% of the formulation, the emollient system including a first component with a refractive index of at least 1.40, a second component, and optionally a third component; a thickener in an amount of 1.5% to 5.0% of the formulation; pigments in an amount of 10.0% to 20.0% of the formulation; an absorbent powder in an amount of 1.5% to 5.0% of the formulation; and an emulsifier in an amount of 2.0% to 8.0% of the formulation; and a water phase in an amount of 10.0% to 25.0% of the formulation.

In some aspects, the techniques described herein relate to a cosmetic formulation, wherein the formulation subsequent to application to a surface demonstrates Longwear of at least 24 hours.

In some aspects, the techniques described herein relate to a cosmetic formulation, wherein the formulation subsequent to application to the surface demonstrates a shine intensity of at least 30 when measured by glossmeter at six hours subsequent to application to the surface.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, formulation, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A cosmetic formulation comprising a water in oil emulsion for application to an integument, wherein the cosmetic formulation comprises:
a discontinuous water phase; and
a continuous oil phase comprising:
a film former comprising an MQ resin in an amount of 5% to 10% of the formulation; and
an emollient system having a first component with a refractive index of at least 1.40;
wherein the formulation subsequent to application to a surface demonstrates Longwear of at least 24 hours, and
wherein the formulation subsequent to application to the surface demonstrates a shine intensity of at least 30 when measured by glossmeter at six hours subsequent to application to the surface.

2. The cosmetic formulation of claim 1, wherein the formulation has a viscosity of 10,000 cP to 20,000 cP as measured via Brookfield viscosity with a TB spindle at 10 rpm for 1 minute.

3. The cosmetic formulation of claim 1, wherein the formulation comprises a stability of at least 8 weeks.

4. The cosmetic formulation of claim 1, wherein the formulation subsequent to application to a surface demonstrates Longwear of at least 36 hours.

5. The cosmetic formulation of claim 1, wherein the formulation subsequent to application to the surface demonstrates a shine intensity of 30 to 50 when measured by glossmeter at six hours subsequent to application to the surface.

6. The cosmetic formulation of claim 1, wherein film former comprises 5% to 10% of the emulsion.

7. The cosmetic formulation of claim 1, wherein film former comprises trimethylsiloxysilicate.

8. The cosmetic formulation of claim 1, wherein the emollient system comprises about 15% to about 45% of the emulsion.

9. The cosmetic formulation of claim 1, wherein the first component of the emollient system comprises 10% to 20% of the emollient system.

10. The cosmetic formulation of claim 1, wherein the first component of the emollient system comprises diphenylsiloxy phenyl trimethicone.

11. The cosmetic formulation of claim 1, wherein the emollient system further comprises diisopropyl sebacate, dimethicone, and combinations thereof.

12. The cosmetic formulation of claim 1, wherein the oil phase further comprises an emulsifier being 2.0% to 8.0% of the formulation.

13. The cosmetic formulation of claim 1, wherein the oil phase further comprises a thickener.

14. The cosmetic formulation of claim 1, wherein the oil phase further comprises one or more pigments including yellow pigments, red pigments, black pigments, white pigments, or combinations thereof.

15. The cosmetic formulation of claim 1, wherein the discontinuous water phase comprises a moisturizing ingredient.

16. The cosmetic formulation of claim 1, further comprising an absorbent powder in an amount of 1.5% to 5.0%.

17. The cosmetic formulation of claim 1, further comprising Nymphaea Caerulea Flower Extract and Nelumbo Nucifera Flower Extract.

18. A cosmetic formulation comprising a water in oil emulsion for application to an integument, wherein the cosmetic formulation comprises:
An oil phase comprising:
a film former comprising an MQ resin, the film former in an amount of 5.0% to 10.0% of the formulation; and
an emollient system having the emollient system being in an amount of 15.0% to 45.0% of the formulation, the emollient system including a first component with a refractive index of at least 1.40, a second component, and optionally a third component;
a thickener in an amount of 1.5% to 5.0% of the formulation;
pigments in an amount of 10.0% to 20.0% of the formulation;
an absorbent powder in an amount of 1.5% to 5.0% of the formulation; and
an emulsifier in an amount of 2.0% to 8.0% of the formulation; and
a water phase in an amount of 10.0% to 25.0% of the formulation.

19. The cosmetic formulation of claim 18, wherein the formulation subsequent to application to a surface demonstrates Longwear of at least 24 hours.

20. The cosmetic formulation of claim 18, wherein the formulation subsequent to application to the surface demonstrates a shine intensity of at least 30 when measured by glossmeter at six hours subsequent to application to the surface.
